(19) United States
(12) Patent Application Publication
(10) Pub. No.: US 2011/0124974 A1
(43) Pub. Date: May 26, 2011

(54) MOBILE MEDICAL COMMUNICATIONS SYSTEM

(76) Inventors: Carlos Edward Martin, Columbia, SC (US); Shirley Ann Martin Flowers, Columbia, SC (US)

(21) Appl. No.: 12/624,549

(22) Filed: Nov. 24, 2009

(51) Int. Cl. A61B 5/00; H04N 7/18

(52) U.S. Cl. 600/300; 348/61; 348/E07.085

(57) ABSTRACT

A mobile medical communications system, having a processor, at least one video capture device, an audio emitting device, an audio receiving device, and a communications device, the system being communicatively coupled to a remote terminal via the communications device. In one embodiment the system may include a patient scanning device, a vital sign measuring and recording device and a global positioning unit.

### BACKGROUND

Emergency medical technicians, paramedics and other primary responders are usually the first medically-trained individuals to arrive on the scene of an accident, disaster, or other medical emergency. Primary responders are well-equipped and trained to provide first-aid and other immediate life-saving medical treatment, such as maintaining the patient's breathing and circulation, cardiopulmonary resuscitation, defibrillation, controlling severe external bleeding, preventing shock, and so on. However, if treatment beyond the advanced life support capabilities is required, such as, for example, in the case of serious injuries, the patient must be transported to a medical center for further treatment.

The transport of a patient to a medical center may be time-consuming, especially when the accident scene is in a remote area. Furthermore, in the case of a major disaster, certain medical centers may be over capacity. As time is critical in emergency situations, communication with the medical center prior to and during patient transport is necessary to provide proper care to the patient as well as to allow the medical center to prepare for the patient's arrival. Additionally, in a mass casualty event, communications between medical centers and primary responders may result in a more efficient distribution of patients among a region's medical centers.

### SUMMARY

According to at least one exemplary embodiment, a mobile medical communications system is provided. The mobile medical communications system may include a processor, at least one video capture device, an audio emitting device, an audio receiving device, and a communications device, the system being communicatively coupled to a remote terminal via the communications device. In one embodiment the system may include a patient scanning device, a vital sign measuring and recording device and a global positioning unit.

According to at least one exemplary embodiment, a method of providing medical treatment to a patient is provided. The method may include establishing a communications connection between a mobile medical communications system and a remote terminal, focusing at least one video capture device on an area of the patient's body that is in need of treatment, displaying video data captured by the at least one video capture device on at least one video display screen of the remote terminal such that said video data is displayed in real time and establishing audio communication between the mobile medical communications system and the remote terminal such that the audio communication is carried out in real time. The method may also include analyzing the patient using a patient scanning device, communicating the obtained data to a remote terminal, and displaying the data on at least one video display screen of the remote terminal.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1a is an exemplary diagram of a mobile medical communications device.

FIG. 1b is an exemplary diagram of a remote terminal for a mobile medical communications device.

FIG. 2a is an exemplary diagram of a video capture device for a mobile medical communications device.

FIG. 2b is an exemplary diagram of an audio emitting device for a mobile medical communications device.

FIG. 2c is an exemplary diagram of an audio receiving device for a mobile medical communications device

FIG. 2d is an exemplary diagram of a vital sign measuring and recording device for a mobile medical communications device.

### DETAILED DESCRIPTION OF THE EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance or illustration.” Any embodiment described herein as “exemplary” is not necessarily to be construed as preferred or advantageous over other embodiments. Likewise, the term “embodiments of the invention” does not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

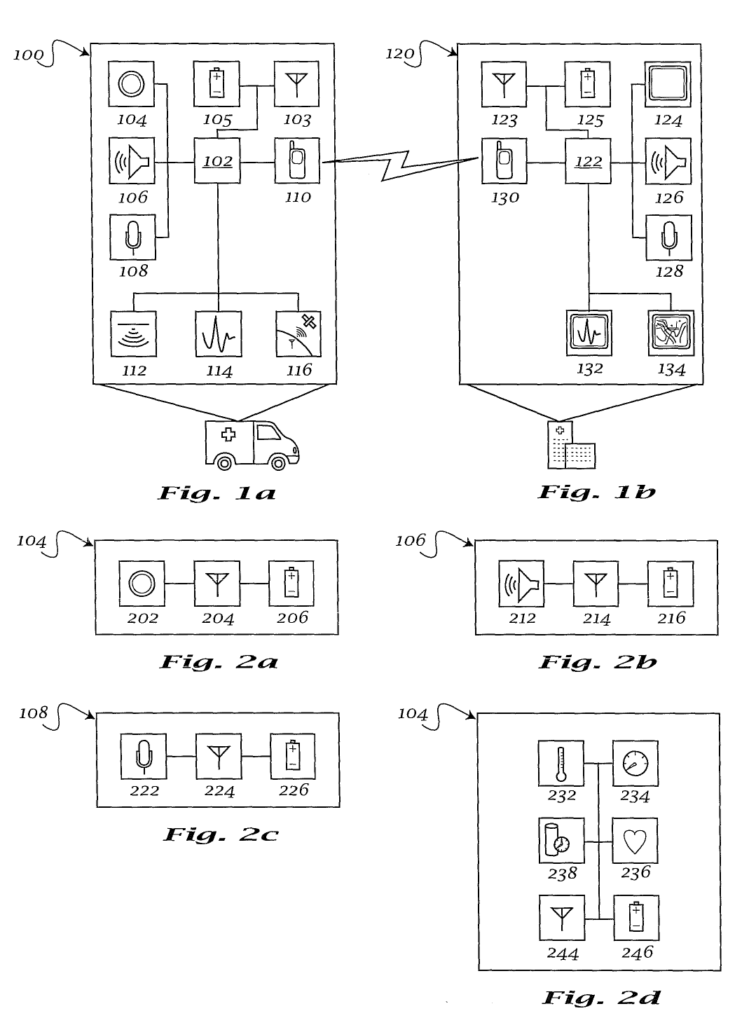

Turning to the accompanying figures, a mobile medical communications system 100 is disclosed. As shown in FIG. 1a, in one embodiment, system 100 may include a processor 102, at least one video capture device 104, an audio emitting device 106, an audio receiving device 108 and a first communications device 110. System 100 may further include a patient scanning device 112, a vital sign measuring and recording device 114, and a global positioning unit 116. In one exemplary embodiment, mobile medical communications system 100 may be integrated into an ambulance or other emergency vehicle, and may include portable wireless components. In another exemplary embodiment, mobile medical communications system 100 may be fully portable and independent of the ambulance or other emergency vehicle. Mobile communications system 100 may also be communicatively coupled to a remote terminal 120. Remote terminal 120 may include a processor 122, at least one video display screen 124, an audio emitting device 126, an audio receiving device 128 and a second communications device 130, as shown in FIG. 1b. Remote terminal 120 may further include a patient vital sign display 132, and a mapping display 134. Remote terminal 120 may be located at a hospital or other medical center or emergency service facility.

Processor 102 may be provided by a microcontroller, as is known to one having ordinary skill in the art. In addition to the conventional arithmetic and logic elements of a conventional general-purpose microprocessor, microcontrollers integrate additional elements, such as read-write memory for data storage, read-only memory, input/output interfaces, timers, drivers and the like. In a non-limiting manner, volatile memory can include RAM for data storage. In a non-limiting manner, non-volatile memory can include ROM, EPROM, EEPROM and Flash memory for program and operating parameter storage. In one exemplary embodiment, processor 102 may be operatively coupled to a power source 105, a transmitter/receiver 103 and first communications device 110. Power source 105 may be, but not limited to, at least one battery, an alternative current power source, a solar power source, and/or any other power that enables processor 102 to function as described herein. Transmitter/receiver 103 may wirelessly communicate with the components of system 100 described herein through a variety of different methods, for example, via Bluetooth technology, infrared, radio frequency, Wi-Fi, wide-band and/or any other type of wireless communication technology that enables mobile medical communications system 100 to function as described herein. Processor 102 may aggregate data from components of system 100 described herein and relay said data to first communications device 110 for transmission to remote terminal 120. Similarly, processor 102 may receive data from communications device 110 and relay it to the appropriate components of system 100 described herein.

Turning to FIG. 2a, at least one video capture device 104 may include a camera lens 202, a transmitter 204 and a power source 206, wherein camera lens 202 may be operatively coupled to transmitter 204 and power source 206 such that camera lens 202 may capture images and transmit the captured images to processor 102. Power source 206 may be, but not limited to, at least one battery, an alternative current power source, a solar power source, and/or any other power that enables at least one video capture device 104 to function as described herein. In one embodiment, camera lens 202 may capture and transmit moving pictures and/or still pictures to processor 102. Transmitter 204 may transmit data to processor 102 wirelessly through a variety of different methods, for example, via Bluetooth technology, infrared, radio frequency, Wi-Fi, wide-band and/or any other type of wireless communication technology that enables mobile medical communications system 100 to function as described herein. In one embodiment, at least one video capture device 104 may be mounted within an emergency vehicle, or may be a standalone unit. In another embodiment, at least one video capture device 104 may be worn by the user or may be part of a headset worn by the user, wherein the headset may also include audio emitting device 106 and audio receiving device 108.

Turning to FIG. 2b, audio emitting device 106 may include an audio speaker 212, a receiver 214 and a power source 216, wherein audio speaker 212 may be operatively coupled to receiver 214 and power source 216 such that audio speaker 212 may receive audio information from processor 102 and emit audio information in human-audible wavelengths via audio speaker 212. Power source 216 may be, but not limited to, at least one battery, an alternative current power source, a solar power source, and/or any other power that enables audio emitting device 106 to function as described herein. Receiver 214 may receive data from processor 102 wirelessly through a variety of different methods, for example, via Bluetooth technology, infrared, radio frequency, Wi-Fi, wide-band and/or any other type of wireless communication technology that enables mobile medical communications system 100 to function as described herein. In one embodiment, audio emitting device 106 may be mounted within an emergency vehicle, or may be a standalone unit. In another embodiment, audio emitting device 106 may be worn by the user or may be part of a headset worn by the user, wherein the headset may also include at least one video capture device 104 and audio receiving device 108.

Turning to FIG. 2c, audio receiving device 108 may include a microphone 222, a transmitter 224 and a power source 226, wherein microphone 222 may be operatively coupled to transmitter 224 and power source 226 such that microphone 222 may detect sound vibrations and transmit the detected data to processor 102. Microphone 222 may be, but not limited to, a parabolic microphone that is capable of filtering ambient noise, or any other ambient noise filtering microphone technology known to one having ordinary skill in the art. Power source 226 may be, but not limited to, at least one battery, an alternative current power source, a solar power source, and/or any other power that enables audio receiving device 108 to function as described herein. Transmitter 224 may transmit data to processor 102 wirelessly through a variety of different methods, for example, via Bluetooth technology, infrared, radio frequency, Wi-Fi, wide-band and/or any other type of wireless communication technology that enables mobile medical communications system 100 to function as described herein. In one embodiment, audio receiving device 108 may be mounted within an emergency vehicle, or may be a standalone unit. In another embodiment, audio receiving device 108 may be worn by the user or may be part of a headset worn by the user, wherein the headset may also include at least one video capture device 104 and audio receiving device 106.

Communications device 110, 130 may be a wireless transmission device configured to utilize one or several of a variety of long-distance wireless communications technologies that support video and audio transmission. For example, communications devices 110, 130 may be configured to utilize CDMA, TDMA, GSM, satellite technology or any other wireless communication technology known to one having ordinary skill in the art. In one embodiment, communications devices 110, 130 may utilize the third generation of wireless technologies (also known as IMT-2000 or 3G) or other technologies that meet or exceed the IMT-2000 standard. Communications device 110 may be communicatively coupled to second communications device 130 of remote terminal 120, thereby facilitating real-time data exchange between mobile medical communications system 100 and remote terminal 120.

In one embodiment, mobile medical communications system 100 may include patient scanning device 112. Patient scanning device may be, for example, a real-time 3D medical imaging scanner, or any other medical scanning device known to one having ordinary skill in the art. Patient scanning device 112 may be operatively coupled to processor 102. Patient scanning device 112 may enable the user to scan areas of the patient's body to provide three-dimensional imaging of the patient's wounds and/or injuries. Data from scanner 112 may then be processed by processor 102 and transmitted to remote terminal 120 via communications devices 110, 130. Data from patient scanning device 112 may then be displayed, for example, as a three dimensional model, on at least one display screen 124 of remote terminal 120.

Turning to FIG. 2d, in one embodiment, mobile medical communications system 100 may also include vital sign measuring and recording device 114. Vital sign measuring and recording device 114 may include, but not limited to, a thermometer 232, a sphygmomanometer 234, a heart rate monitor 236, and a respirometer 238. Vital sign measuring and recording device 114 may also include a transmitter 244 and a power source 246, wherein thermometer 232, sphygmomanometer 234, heart rate monitor 236, and respirometer 238 may be operatively coupled to transmitter 244 and power source 246 such that patient vital signs may be transmitted to processor 102. Power source 246 may be, but not limited to, at least one battery, an alternative current power source, a solar power source, and/or any other power that enables vital sign measuring and recording device 114 to function as described herein. Transmitter 244 may transmit data to processor 102 wirelessly through a variety of different methods, for example, via Bluetooth technology, infrared, radio frequency, Wi-Fi, wide-band and/or any other type of wireless communication technology that enables mobile medical communications system 100 to function as described herein. Data from vital sign measuring and recording device 114 may then be processed by processor 102 and transmitted to remote terminal 120 via communications devices 110, 130. Data from vital sign measuring and recording device 114 may then be displayed on patient vital sign display 132 of remote terminal 120.

Referring back to FIGS. 1a and 1b, in one embodiment, mobile medical communications system 100 may include global positioning unit 116. Global positioning unit 116 may be operatively coupled to processor 102. Data from global positioning unit 116 may then be processed by processor 102 and transmitted to remote terminal 120 via communications devices 110, 130. Global positioning data from global positioning unit may then be displayed on mapping display 134 of remote terminal 120. The remote terminal user may thus be able to determine the location of the ambulance or other emergency vehicle that is utilizing mobile medical communications system 100.

In operation, a first responder may utilize mobile medical communications system 100 when treating a patient. The first responder may establish a connection between system 100 and remote terminal 120 and may focus at least one video capture device 104 on the area of the patient's body that is in need of treatment. Data captured by at least one video capture device 104 may then be displayed, in real time, on at least one display screen 124 of remote terminal 120 so that a medical professional at remote terminal 120 may observe the condition and treatment of the patient. The first responder and medical professional may then communicate with each other, in real time, utilizing audio emitting device 106 and audio receiving device 108 of system 100 and audio emitting device 126 and audio receiving device 128 of remote terminal 120, respectively. System 100 and remote terminal 120 may thus improve communication between the first responder and the medical professional by presenting both audio and visual means of communication.

In one embodiment, the first responder may utilize patient scanning device 112 and vital sign measuring and recording device 114 to further analyze the condition of the patient. Data captured by patient scanning device 112 may then be displayed on at least one display screen 124 of remote terminal 120 so that the medical professional at remote terminal 120 may observe the condition and treatment of the patient. Data captured by vital sign measuring and recording device 114 may then be displayed on patient vital sign display 132 of remote terminal 120 so that the medical professional at remote terminal 120 may observe the vital signs of the patient. In one embodiment, vital sign data captured by vital sign measuring and recording device 114 may be displayed on at least one display screen 124 of remote terminal 120.

In one embodiment, global positioning data received by global positioning unit 116 may be transmitted to remote terminal 120 and displayed on mapping display 134. A medical professional or other user of remote terminal 120 may then see the position of system 100, or a plurality of systems 100 on mapping display 134. Ambulances or other emergency vehicles may then be directed to a particular medical center or other emergency facility such that patients may receive the necessary care in an expedient manner.

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A mobile medical communications system, comprising:
a processor;
at least one video capture device;
an audio emitting device;
an audio receiving device; and
a communications device, that communicatively couples the system to a remote terminal.

2. The mobile medical communications system of claim 1, further comprising:
a patient scanning device;
a vital sign measuring and recording device; and
a global positioning unit.

3. The mobile medical communications system of claim 1, wherein said remote terminal comprises:
a processor;
at least one video display screen;
an audio emitting device;
an audio receiving device; and
a communications device.

4. The mobile medical communications system of claim 1, wherein said mobile medical communications system is integrated into an emergency vehicle.

5. The mobile medical communications system of claim 1, wherein said mobile medical communications system is portable.

6. A method of providing medical treatment to a patient, comprising:
establishing a communications connection between a mobile medical communications system and a remote terminal;
focusing at least one video capture device on an area of the patient's body in need of treatment;
displaying video data captured by said at least one video capture device on at least one video display screen of said remote terminal such that said video data is displayed in real time; and
establishing audio communication between said mobile medical communications system and said remote terminal such that said audio communication is carried out in real time.

7. The method of claim 6, further comprising:
analyzing the patient using a patient scanning device;
communicating the obtained data to a remote terminal; and
displaying said data on at least one video display screen of said remote terminal.

8. The method of claim 6, further comprising:
measuring the patient's vital signs using a vital sign measuring and recording device;
communicating the obtained data to a remote terminal; and
displaying said data on a vital sign display of a remote terminal.

9. The method of claim 6, further comprising:
determining the location of an emergency vehicle using a global positioning unit;
communicating the obtained data to a remote terminal; and
displaying the location of the emergency vehicle on a mapping display of the remote terminal.
